(19) United States
(12) Patent Application Publication
(10) Pub. No.: US 2002/0132737 A1
(43) Pub. Date: Sep. 19, 2002

(54) Alkoxylated phenol derivatives

(76) Inventors: Helmut-Martin Meier, Ratingen (DE); Hans-Joachim Knackmuss, Leonberg (DE); Paul-Gerhard Rieger, Stuttgart (DE)

Correspondence Address: BAYER CORPORATION, PATENT DEPARTMENT, 100 BAYER ROAD, PITTSBURGH, PA, 15205, US

(21) Appl. No.: 09/942,513

(22) Filed: Aug. 29, 2001

(30) Foreign Application Priority Data
Aug. 31, 2000 (DE) 10042730.8

(51) Int. Cl. A01N 25/00; C09B 67/00; C07C 43/205

(52) U.S. Cl. 504/358; 8/610; 568/608; 514/772

(57) ABSTRACT

The invention relates to compounds of the formula (I)

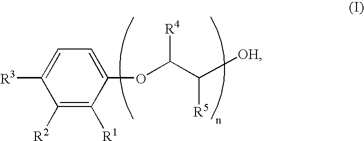

where

R¹, R², and R³ are independently hydrogen, optionally substituted cyclohexyl, or optionally substituted phenyl, with the proviso that at least one of R¹ to R³ is not hydrogen,

R⁴ and R⁵ are independently methyl or hydrogen, and

n is from 21 to 40,

which are biodegradable dispersants having good dispersing properties and are used for dyeing with disperse dyes.

### BACKGROUND OF THE INVENTION

The invention relates to alkoxylated phenol derivatives, a process for their preparation, and their use as dispersants in the dyeing process of textiles.

Disperse dyes are generally water-insoluble or sparingly water-soluble compounds that cannot be used for the dyeing of textiles without the use of a dispersant. The literature describes numerous dispersants that can be used for that purpose. Particularly effective dispersants in this context are ligninsulfonates and naphthalenesulfonates, as documented for example in Chwala/Anger Handbuch der Textilhilfsmittel, 1977, pages 501 ff.

Similarly, alkoxylated phenols and derivatives thereof have already been used for this purpose.

FR-A 2,472,627 explicitly discloses for example ethoxylated phenol derivatives having up to 20 ethylene oxide (“EO”) units (Examples 1, 2, 3, and 5).

GB-A 2,050,438 explicitly discloses ethoxylated p-phenylphenol having 12 EO units (Example 8).

U.S Pat. No. 4,328,220 likewise explicitly discloses ethoxylated phenol derivatives having 8 EO units (compound B 30, column 9, line 9).

But all these alkoxylated phenol derivatives have inadequate dispersing effects.

It is an object of the present invention to provide improved dispersants. It is a further object to provide dispersants possessing good biodegradability, since dispersants from dyeing processes remain in the effluent liquor of the dyeing process and are generally costly to dispose of properly.

### SUMMARY OF THE INVENTION

These and other objects have surprisingly been achieved with compounds of the formula (I)

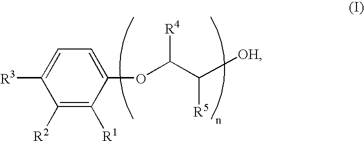

where

R¹, R², and R³ are independently hydrogen, optionally substituted cyclohexyl, or optionally substituted phenyl, with the proviso that at least one of R¹ to R³ is not hydrogen,

R⁴ and R⁵ are independently methyl or hydrogen, and

n is from 21 to 40.

### DETAILED DESCRIPTION OF THE INVENTION

Possible substituents of the cyclohexyl radical include, for example, C₁-C₄-alkyl, C₁-C₄-alkoxy, halogen, CN, or nitro. Preferably, however, the cyclohexyl radical is unsubstituted.

Possible substituents for the phenyl radical include for example C₁-C₄-alkyl, C₁-C₄-alkoxy, halogen, CN, or NO₂. Preferably, however, the phenyl radical is unsubstituted.

The value of n is preferably from 26 to 35, particularly preferably from 29 to 31.

Particularly preferred phenol radicals of the formula

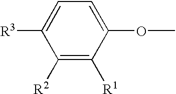

include, for example, those which are derived from the corresponding phenols of the formula (II), for example, those from the group consisting of o-cyclohexylphenol, m-cyclohexylphenol, p-cyclohexylphenol, o-phenyl-phenol, m-phenylphenol, and p-phenylphenol. Particular preference is given to o-phenylphenol and p-phenylphenol, especially p-phenylphenol.

R⁴ and R⁵ are both particularly preferably hydrogen.

Very particular preference is given to the compound of the formula

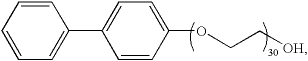

which is a very good dispersant and also readily biodegradable.

The invention further relates to a process for preparing the compounds of the formula (I) according to the invention comprising reacting phenols of the formula (II)

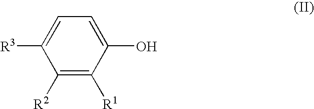

where R¹, R², and R³ are defined as for formula (I),

with n mol equivalents of an alkylene oxide of the formula (III)

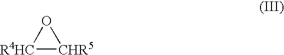

where R⁴, R⁵, and n are defined as for formula (I).

Useful alkylene oxides of the formula (III) preferably include ethylene oxide and/or propylene oxide, especially ethylene oxide.

The alkoxylation is preferably effected in the presence of alkali or alkaline earth metal catalysts, these preferably being used in an amount of 0.1 to 1% by weight, based on the phenol derivative of the formula (II).

Particularly suitable compounds of the formula (II) are o-cyclo-hexylphenol, m-cyclohexylphenol, p-cyclohexylphenol, o-phenylphenol, m-phenylphenol, and p-phenylphenol.

The reaction of the phenol derivatives of the formula (II) with the alkylene oxides of the formula (III), especially ethylene oxide or propylene oxide, is generally carried out by known processes and is described, for example, in N. Schönfeldt, Grenzflächenaktive Ethylenoxid-Addukte, Stuttgart, 1976.

The invention relates to the use of the compounds according to the invention as dispersants for formulations containing pharmaceutical or agrochemical active compounds as well as to the corresponding formulations.

The invention further provides for the use of the compounds according to the invention as dispersants in dyeing or printing with disperse dyes and also in the dyeing of textiles, especially in the dyeing of hydrophobic fibers.

Several known processes are available for dyeing hydrophobic fibers. They are described, for example, in M. Peter, H. K. Rouette, Grundlagen der Textilveredlung, Frankfurt, 1989, section 7.2.2. Particular preference is given to using dispersants in the dyeing of polyester fibers and their blends, for example with wool, cotton, polyamide, and acrylic.

The compounds of the formula (I) may advantageously be used in the carrier dyeing process, in the HT dyeing process, and also in the thermosol dyeing process. In the HT dyeing process, the bath, which has been heated to 50-60° C. and preferably contains 1-2 g/l of compound of the formula (II), is generally adjusted to pH 4.5-5 with acetic acid and admixed with one or more disperse dyes according to the depth of shade and the liquor ratio. The liquor is then preferably introduced into the dyeing machine together with the material to be treated. This is preferably followed by heating to 130° C. over 30 to 60 minutes under pressure and dyeing at 130° C. for 60 to 90 minutes. The dyeing is customarily followed by a reductive aftertreatment with 2 g/l of sodium hydrosulfite at 75-85° C. for 20 minutes. Use of the compound of the formula (I) according to the invention provides excellent fastness. It is likewise possible to use further customary auxiliaries such as dispersants, leveling agents, carriers, and aftertreatment agents in the dyeing.

The compounds of the formula (I) according to the invention can be used in pure form or in the form of aqueous solutions with or without customary solvents such as glycol or polyglycol. A preferred form is the aqueous solution of compounds of the formula (I) in a concentration of 10 to 90% by weight, preferably 70 to 30% by weight, particularly preferably 45 to 35% by weight. The solutions can be prepared for example by mixing the compounds of the invention with solvents in suitable stirred apparatuses, for example, at room temperature or with heating.

The following examples further illustrate details for the preparation and use of the compounds of this invention. The invention, which is set forth in the foregoing disclosure, is not to be limited either in spirit or scope by these examples. Those skilled in the art will readily understand that known variations of the conditions and processes of the following preparative procedures can be used to prepare these compounds. Unless otherwise noted, all temperatures are degrees Celsius and all percentages are percentages by weight.

### EXAMPLES

The dispersion properties were tested by means of a dispersant test described below.

The biodegradability was measured using a test according to OECD 301 A ISO 7827 (DIN 7827) (measurement of the decrease in organically bound carbon).

Under these guidelines, an organic substance is classed as “readily biodegradable” in the OECD 301 A to F test when the degradation amounts to >60%.

Dispersant Test

5.0 g of Resolin Blue BBLS (C.l. Disperse Blue 56) and 10.0 g of sodium acetate (trihydrate) were bulked with demineralized water to 5,000 ml. During stirring, the pH was adjusted to 4.5 with acetic acid.

100 ml lots of this dye liquor were used together with 1 g/l of the dispersant to be tested. The resultant liquor was subsequently subjected to a 120° C. dyeing program on a Spectra Dye machine (pot dyeing). The liquor was then filtered through a 5.5 cm glass slotted sieve suction filter (with 3 S&S BF filter discs).

A 1 ml sample was taken from both the liquor prior to filtration and from the filtrate and dissolved in 20 ml of acetone. The dye quantities contained in the acetonic solutions were evaluated in UV-VIS (1 cm cell, 610 nm). The relevant quantity is considered to be the dispersion factor (“DF”), which is the absorbance of filtrate/absorbance of made-up liquor. A DF of 1 indicates a very good dispersant, while a DF of 0 indicates a dispersant lacking utility.

### Preparation Example 1

170.2 g of p-phenylphenol (1 mol) were dewatered at 170° C. in an autoclave in the course of 10 minutes under a high vacuum and then reacted at 2.5 bar/170° C. with 1 320 g of ethylene oxide (30 mol) in the presence of 1.7 g of potassium hydroxide over 10 hours. The end product was used in the form of an aqueous solution as a dispersant without further work-up. The properties with regard to biodegradability and dispersing power are shown in the table.

### Examples 2 to 4 (Inventive)

Further compounds according to the invention were prepared similarly to Example 1, except that the reaction temperature was varied from 120° C. to 170° C. and the pressure from 2.0 to 4 bar depending on the starting material. The properties with regard to biodegradability and dispersing power are shown in the table.

### Comparative Examples 1 and 2

Ethoxylated p-phenylphenol (Example 3 of FR-A 2,472,627; 20 mol equivalent of EO) and o-phenylphenol (Example 2 of FR-A 2,472,627; 15 mol equivalent of EO) were prepared and compared with Inventive Examples 1 and 2 respectively. Surprisingly, the compounds according to the invention had a substantially higher DF value.

TABLE

| | | Reaction product | Biode- | |
|---|---|---|---|---|
| | | with n mol | gradability | |
| | Phenol derivative of | equivalent of | to OECD | |
| Example | formula (II) | ethylene oxide | 301 A | DF |
| Inventive 1 | p-Phenylphenol | 30 | 94% | 1.0 |
| Compara- | p-Phenylphenol | 20 | 98% | 0.3 |
| tive 1 | | | | |
| Inventive 2 | o-Phenylphenol | 26 | 67% | 0.9 |
| Compara- | o-Phenylphenol | 15 | 67% | 0.5 |
| tive 2 | | | | |
| Inventive 3 | o-Cyclohexylphenol | 21 | 74% | 0.9 |
| Inventive 4 | p-Cyclohexylphenol | 21 | 68% | 0.9 |

### Use Example

A drum dyeing machine having a capacity of 800 liters was used to dye 90 kg of polyester slubbing with a mixture of 70 g of Teratop Yellow GWL 150% (C.I. constitution No. 10 338), 31 g of Teratop Blue BLF (C.I. Disperse Blue 62), 3,000 g of Terasil Red R (C.I. Disperse Red 324), and 2,000 g of Fantagen Bordeaux 3BL at 130° C. The dispersant is used in an amount of 1 g/l.

The dispersants used were the compounds of Inventive Example 3 and Inventive Example 1. Dyeings having excellent washfastness at 50° C., perspiration fastness, and very good alkaline setting fastness at 180° C. were obtained in both cases.

What is claimed is:

1. A compound of the formula (I) 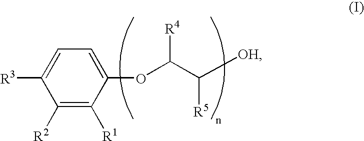
where
R¹, R², and R³ are independently hydrogen, optionally substituted cyclohexyl, or optionally substituted phenyl, with the proviso that at least one of R¹ to R³ is not hydrogen,
R⁴ and R⁵ are independently methyl or hydrogen, and
n is from 21 to 40.

2. A compound according to claim 1 wherein n is from 26 to 35.

3. A compound according to claim 1 wherein R³ is phenyl.

4. A compound according to claim 1 wherein at least one of R¹ to R³ is cyclohexyl and n is from 21 to 40.

5. A process for preparing compounds according to claim 1 comprising reacting phenols of the formula (II) 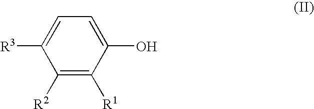
wherein
R¹, R², and R³ are independently hydrogen, optionally substituted cyclohexyl, or optionally substituted phenyl, with the proviso that at least one of R¹ to R³ is not hydrogen
with n mol equivalents of an alkylene oxide of the formula (III) 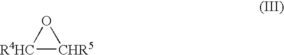
wherein
R⁴ and R⁵ are independently methyl or hydrogen, and
n is from 21 to 40.

6. A method comprising dyeing or printing with disperse dyes in the presence of compounds according to claim 1 as dispersants.

7. A method comprising dyeing textiles in the presence of compounds according to claim 1 as dispersants.

8. A method comprising dyeing hydrophobic fibers in the presence of compounds according to claim 1 as dispersants.

9. Formulations containing pharmaceutical or agrochemical active compounds and dispersants comprising compounds according to claim 1.
